(19) United States
(12) Patent Application Publication
(10) Pub. No.: US 2009/0124640 A1
(43) Pub. Date: May 14, 2009

(54) Pyrrolo[3,2-D]Pyrimidin-4-One Derivative as Myeloperoxidase Inhibitor

(75) Inventors: Anna-Karin Tiden, Sodertalje (SE); Cecilia Weistrand, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/301,050

(22) PCT Filed: Jun. 4, 2007
(86) PCT No.: PCT/SE2007/000538

§ 371 Date: Nov. 17, 2008

Related U.S. Application Data
(60) Provisional application No. 60/810,997, filed on Jun. 5, 2006.

(51) Int. Cl. A61K 31/519; C07D 487/04

(52) U.S. Cl. 514/265.1; 544/280

(57) ABSTRACT

The present invention relates to a new compound A: [Chemical formula should be inserted here. Please see paper copy] a process for its preparation, pharmaceutical formulations containing said therapeutically active compound and to the use of said active compound in therapy. The compound is an inhibitor of the enzyme MPO and is thereby particularly useful in the treatment or prophylaxis of neuroinflammatory disorders, cardiovascular disorders and respiratory disorders.

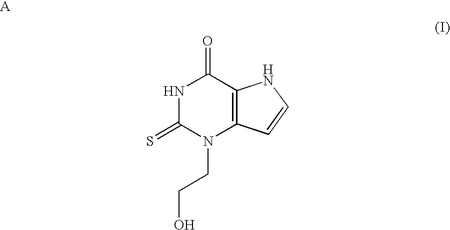

### FIELD OF THE INVENTION

The present invention relates to a novel pyrrolo[3,2-d]pyrimidin-4-one derivative, compositions containing them and their use in therapy.

### BACKGROUND OF THE INVENTION

Myeloperoxidase (MPO) is a heme-containing enzyme found predominantly in polymorphonuclear leukocytes (PMNs). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of identical halves. Each half molecule contains a covalently bound heme that exhibits unusual spectral properties responsible for the characteristic green colour of MPO. Cleavage of the disulphide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. Other halides and pseudohalides (like thiocyanate) are also physiological substrates to MPO.

PMNs are of particular importance for combating infections. These cells contain MPO, with well-documented microbicidal action. PMNs act non-specifically by phagocytosis to engulf microorganisms, incorporate them into vacuoles, termed phagosomes, which fuse with granules containing myeloperoxidase to form phagolysosomes. In phagolysosomes the enzymatic activity of the myeloperoxidase leads to the formation of hypochlorous acid, a potent bactericidal compound. Hypochlorous acid is oxidizing in itself, and reacts most avidly with thiols and thioethers, but also converts amines into chloramines, and chlorinates aromatic amino acids. Macrophages are large phagocytic cells which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple sclerosis, Alzheimer's disease, Parkinson's disease and stroke as well as other inflammatory diseases or conditions like asthma, chronic obstructive pulmonary disease, cystic fibrosis, atherosclerosis, ischemic heart disease, heart failure, inflammatory bowel disease, renal glomerular damage and rheumatoid arthritis. Lung cancer has also been suggested to be associated with high MPO levels.

### Multiple Sclerosis (MS)

MPO positive cells are immensely present in the circulation and in tissue undergoing inflammation. More-specifically MPO containing macrophages and microglia has been documented in the CNS during disease; multiple sclerosis (Nagra R M, et al. Journal of Neuroimmunology 1997; 78(1-2):97-107), Parkinson's disease (Choi D-K. et al. J. Neurosci. 2005; 25(28):6594-600) and Alzheimer's disease (Green P S. et al. Journal of Neurochemistry. 2004; 90(3):724-33). It is supposed that some aspects of a chronic ongoing inflammation result in an overwhelming destruction where agents from MPO reactions have an important role.

The enzyme is released both extracellularly as well as into phagolysosomes in the neutrophils (Hampton M B, Kettle A J, Winterbourn C C. Blood 1998; 92(9): 3007-17). A prerequisite for the MPO activity is the presence of hydrogen peroxide, generated by NADPH oxidase and a subsequent superoxide dismutation. The oxidized enzyme is capable to use a plethora of different substrates of which chloride is most recognized. From this reaction the strong non-radical oxidant—hypochlorous acid (HOCl)— is formed. HOCl oxidizes sulphur containing amino acids like cysteine and methionine very efficiently (Peskin A V, Winterbourn C C. Free Radical Biology and Medicine 2001; 30(5): 572-9). It also forms chloramines with amino groups, both in proteins and other biomolecules (Peskin A V. et al. Free Radical Biology and Medicine 2004; 37(10):1622-30). It chlorinates phenols (like tyrosine) (Hazen S L. et al. Mass Free Radical Biology and Medicine 1997; 23(6): 909-16) and unsaturated bonds in lipids (Albert C J. et al. J. Biol. Chem. 2001; 276(26): 23733-41), oxidizes iron centers (Rosen H, Klebanoff S J. Journal of Biological Chemistry 1982; 257(22): 13731-354) and crosslinks proteins (Fu X, Mueller D M, Heinecke J W. Biochemistry 2002; 41(4): 1293-301).

Proteolytic cascades participate both in cell infiltration through the BBB as well as the destruction of BBB, myelin and nerve cells (Cuzner M L, Opdenakker G. Journal of Neuroimmunology 1999; 94(1-2): 1-14; Yong V W. et al. Nature Reviews Neuroscience 2001; 2(7):5 02-11). Activation of matrix metalloproteinases (MMPs) can be accomplished through the action of upstream proteases in a cascade as well as through oxidation of a disulfide bridge Pu X. et al. J. Biol. Chem. 2001; 276(44): 41279-87; Gu Z. et al. Science 2002; 297(5584): 1186-90). This oxidation can be either a nitrosylation or HOCl-mediated oxidation. Both reactions can be a consequence of MPO activity. Several reports have suggested a role for MMP's in general and MMP-9 in particular as influencing cell infiltration as well as tissue damage (BBB breakdown and demyelination), both in MS and EAE (for review see Yong V W. et al, supra). The importance of these specific kinds of mechanisms in MS comes from studies where increased activity and presence of proteases have been identified in MS brain tissue and CSF. Supportive data has also been generated by doing EAE studies with mice deficient in some of the proteases implicated to participate in the MS pathology, or by using pharmacological approaches.

The demyelination is supposed to be dependent on the cytotoxic T-cells and toxic products generated by activated phagocytes (Lassmann H. J Neurol Neurosurg Psychiatry 2003; 74(6): 695-7). The axonal loss is thus influenced by proteases and reactive oxygen and nitrogen intermediates. When MPO is present it will obviously have the capability of both activating proteases (directly as well as through disinhibition by influencing protease inhibitors) and generating reactive species.

### Chronic Obstructive Pulmonary Disease (COPD)

Chronic obstructive pulmonary disease (COPD) is a disease state characterised by airflow limitation that is not fully reversible. The airflow limitation is usually both progressive and associated with an abnormal inflammatory response of the lungs to noxious particles or gases. COPD is a major public health problem. It is the fourth leading cause of chronic morbidity and mortality in the United States and is projected to rank fifth in 2020 as a worldwide burden of disease. In the UK the prevalence of COPD is 1.7% in men and 1.4% in women. COPD spans a range of severity from mild to very severe, with the cost of treatment rising rapidly as the severity increases.

Levels of MPO in sputum and BAL are much greater in COPD patients than normal, non-smoking controls (Keatings V. M., Barnes P. J. Am. J Respir Crit. Care Med 1997; 155:449-453; Pesci, A. et al. Eur Respir J 1998; 12:380-386). MPO levels are further elevated during exacerbations of the disease (Fiorini G. et al. Biomedicine & Pharmacotherapy 2000; 54:274-278; Crooks S. W. et al. European Respiratory Journal. 15(2): 274-80, 2000). The role of MPO is likely to be more important in exacerbations of COPD (Sharon S. D. et al. Am J Respir Crit. Care Med. 2001; 163: 349-355).

In addition to the destructive capacity of MPO there is a strong clinical link with vascular disease (Baldus S. et al. Circulation 2003; 108: 1440-5). Dysfunctional MPO polymorphisms are associated with a reduced risk of mortality from coronary artery disease (Nikpoor B. et al. Am Heart J 2001; 142: 336), and patients with high serum levels of MPO have increased risk of acute coronary syndrome. The effects of MPO on vascular disease may extend to COPD, since there is strong evidence that the pulmonary vasculature is one of the earliest sites of involvement in the smokers' lung. Striking changes in the intima of the pulmonary arteries have been described which show a dose relationship with smoking (Hale K. A., Niewoehner D. E., Cosio M. G. Am Rev Resp Dis 1980; 122: 273-8). The physiological function of MPO is associated with innate host defence. This role, however, is not critical as most cases of MPO deficient patients have relatively benign symptoms (Parry M. F. et al. Ann Int Med. 1981; 95: 293-301, Yang, K. D., Hill, H. R. Pediatr Infect Dis J. 2001; 20: 889-900). In summary, there is considerable evidence that elevated MPO levels in COPD may contribute to the disease via several mechanisms. A selective inhibitor of MPO would therefore be expected to alleviate both the acute and chronic inflammatory aspects of COPD and may reduce the development of emphysema.

### Atherosclerosis

An MPO inhibitor should reduce the atherosclerotic burden and/or the vulnerability of existing atherosclerotic lesions and thereby decrease the risk of acute myocardial infarction, unstable angina or stroke, and reduce ischemia/reperfusion injury during acute coronary syndrome and ischemic cerebrovascular events. Several lines of data support a role for MPO in atherosclerosis. MPO is expressed in the shoulder regions and necrotic core of human atherosclerotic lesions and active enzyme has been isolated from autopsy specimens of human lesions (Daugherty, A. et al. (1994) J Clin Invest 94(1): 437-44). In eroded and ruptured human lesions, as compared to fatty streaks, an increased number of MPO expressing macrophages have been demonstrated, suggesting a particular role for MPO in acute coronary syndromes (Sugiyama, S. et al. (2001) Am J Pathol 158(3): 879-91). Patients with established coronary artery disease have higher plasma and leukocyte MPO levels than healthy controls (Zhang, R. et al. (2001) Jama 286(17): 2136-42). Moreover, in two large prospective studies plasma levels of MPO predicted the risk of future coronary events or revascularisation (Baldus, S. et al. (2003) Circulation 108(12): 1440-5; Brennan, M. et al. (2003) N Engl J Med 349(17): 1595-604). Total MPO deficiency in humans has a prevalece prevalence of 1 in 2000-4000 individuals. These is individuals appear principally healthy but a few cases of severe Candida infection have been reported. Interestingly, MPO deficient humans are less affected by cardiovascular disease than controls with normal MPO levels (Kutter, D. et al. (2000) Acta Haematol 104(1)). A polymorphism in the MPO promoter affects expression leading to high and low MPO expressing individuals. In three different studies the high expression genotype has been associated with an increased risk of cardiovascular disease (Nikpoor, B. et al. (2001) Am Heart J 142(2): 336-9; Makela, R., P. J. Karhunen, et al. (2003) Lab Invest 83(7): 919-25; Asselbergs, F. W., et al. (2004) Am J Med 116(6): 429-30). Data accumulated during the last ten years indicate that the proatherogenic actions of MPO include oxidation of lipoproteins, induction of endothelial dysfunction via consuming nitric oxide and destabilisation of atherosclerotic lesions by activation of proteases (Nicholls, S. J. and S. L. Hazen (2005) Arterioscler Thromb Vasc Biol 25(6): 1102-11). Recently, several studies have focused on nitro- and chlorotyrosine modifications of LDL and HDL lipoproteins. Since chlorotyrosine modifications in vivo only can be generated by hypochlorus acid produced by MPO these modifications are regarded as specific markers of MPO activity (Hazen, S. L. and J. W. Heinecke (1997) J Clin Invest 99(9): 2075-81). LDL particles exposed to MPO in vitro become aggregated, leading to facilitated uptake via macrophage scavenger receptors and foam cell formation (Hazell, L. J. and R. Stocker (1993) Biochem J 290 (Pt 1): 165-72). Chlorotyrosine modification of apoA1, the main apolipoprotein of HDL cholesterol, results in impaired cholesterol acceptor function (Bergt, C., S. et al. (2004) Proc Natl Acad Sci USA; Zheng, L. et al. (2004) J Clin Invest 114(4): 529-41). Systematic studies of these mechanisms have shown that MPO binds to and travels with apoA1 in plasma. Moreover, MPO specifically targets those tyrosine residues of apoA1 that physically interact with the macrophage ABCA1 cassette transporter during cholesterol efflux from the macrophage (Bergt, C. et al. (2004) J Biol Chem 279(9): 7856-66; Shao, B. et al. (2005) J Biol Chem 280(7): 5983-93; Zheng et al. (2005) J Biol Chem 280(1): 38-47). Thus, MPO seems to have a dual aggravating role in atherosclerotic lesions, i.e. increasing lipid accumulation via aggregation of LDL particles and decreasing the reverse cholesterol transport via attack on the HDL protein apoA1.

The present invention discloses novel thioxanthine that displays useful properties as inhibitors of the enzyme MPO. Furthermore, the novel compound of the present invention display either one or more than one of the following: (i) improved selectivity towards TPO; (ii) unexpectedly high inhibitory activity towards MPO; (iii) improved brain permeability; (iv) improved solubility and/or (v) improved half-life; when compared to known thioxanthines. Such thioxanthines are disclosed in e.g. WO 03/089430 and WO 05/037835.

### DISCLOSURE OF THE INVENTION

The present invention provides the following Compound A:

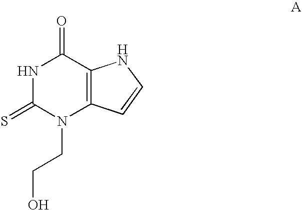

or a pharmaceutically acceptable salt, solvate or solvate of a salt thereof.

For the avoidance of doubt it is to be understood that where in this specification a group is qualified by “hereinbefore defined”, “defined hereinbefore”, “is as defined above” or “are as defined above”, the said group encompasses the first occurring and broadest definition as well as each and all of the preferred definitions for that group.

The present invention relates to the use of Compound A as hereinbefore defined as well as the use of the salts thereof. Salts for use in pharmaceutical compositions will be pharmaceutically acceptable salts, but other salts may be useful in the production of compound A.

The present invention includes a compound A and also in its form of a salt. Suitable salts include those formed with organic or inorganic acids or organic or inorganic bases. Such salts will normally be pharmaceutically acceptable although salts of non-pharmaceutically acceptable acids or bases may be of utility in the preparation and purification of the compound in question. Thus, acid addition salts include inter alia those formed from hydrochloric acid. Base addition salts include those in which the cation is inter alia sodium or potassium.

Compound A may exist in isolated form or in essentially pure form.

A further aspect of the invention is a compound A, or a pharmaceutically acceptable salt thereof, for use as a medicament.

A further aspect of the present invention is the use of a compound A, or a pharmaceutically acceptable salt thereof or solvate or solvate of a salt thereof, in the manufacture of a medicament, for the treatment or prophylaxis of diseases or conditions in which inhibition of the enzyme MPO is beneficial.

A further aspect of the present invention provides the use of a compound A, or a pharmaceutically acceptable salt thereof or solvate or solvate of a salt thereof, in the manufacture of a medicament, for the treatment or prophylaxis of neuroinflammatory disorders, cardio- and cerebrovascular atherosclerotic disorders and peripheral artery disease, heart failure and respiratory disorders such as chronic obstructive pulmonary disease (COPD).

Another further aspect of the present invention provides the use of a compound A, or a pharmaceutically acceptable salt thereof or solvate or solvate of a salt thereof, in the manufacture of a medicament, for the treatment or prophylaxis of multiple sclerosis. Treatment may include slowing progression of disease.

Another further aspect of the present invention provides the use of a compound A, or a pharmaceutically acceptable salt thereof or solvate or solvate of a salt thereof, in the manufacture of a medicament, for the treatment or prophylaxis of Parkinson's disease. Treatment may include slowing progression of disease.

Another further aspect of the present invention provides the use of a compound A or a pharmaceutically acceptable salt thereof or solvate or solvate of a salt thereof, in the manufacture of a medicament, for the treatment or prophylaxis of atherosclerosis by preventing and/or reducing the formation of new atherosclerotic lesions or plaques and/or by preventing or slowing progression of existing lesions and plaques.

Another further aspect of the present invention provides the use of a compound A or a pharmaceutically acceptable salt thereof or solvate or solvate of a salt thereof, in the manufacture of a medicament, for the treatment or prophylaxis of atherosclerosis by changing the composition of the plaques to reduce the risk of plaque rupture and atherothrombotic events.

Another further aspect of the present invention provides the use of a compound A or a pharmaceutically acceptable salt thereof or solvate or solvate of a salt thereof, in the manufacture of a medicament, for the treatment or prophylaxis of respiratory disorders, such as chronic obstructive pulmonary disease. Treatment may include slowing progression of disease.

According to the present invention, there is also provided a method of treating, or reducing the risk of, diseases or conditions in which inhibition of the enzyme MPO is beneficial which comprises administering to a person suffering from or at risk of, said disease or condition, a therapeutically effective amount of a compound A, or a pharmaceutically acceptable salt thereof or solvate or solvate of a salt thereof.

Further, there is also provided a method of treating, or reducing the risk of, neuroinflammatory disorders, cardio- and cerebrovascular atherosclerotic disorders or peripheral artery disease, or heart failure or respiratory disorders, such as chronic obstructive pulmonary disease (COPD), in a person suffering from or at risk of, said disease or condition, wherein the method comprises administering to the person a therapeutically effective amount of a compound A, or a pharmaceutically acceptable salt thereof or solvate or solvate of a salt thereof.

Further, there is also provided a method of treating, or reducing the risk of, multiple sclerosis in a person suffering from or at risk of, said disease or condition, wherein the method comprises administering to the person a therapeutically effective amount of a compound A, or a pharmaceutically acceptable salt thereof or solvate or solvate of a salt thereof.

Further, there is also provided a method of treating, or reducing the risk of, Parkinson's disease in a person suffering from or at risk of, said disease or condition, wherein the method comprises administering to the person a therapeutically effective amount of a compound A, or a pharmaceutically acceptable salt thereof or solvate or solvate of a salt thereof.

There is also provided a method of treating, or reducing the risk of atherosclerosis by preventing and/or reducing the formation of new atherosclerotic lesions or plaques and/or by preventing or slowing progression of existing lesions and plaques in a person suffering from or at risk of, said disease or condition, wherein the method comprises administering to the person a therapeutically effective amount of a compound A or a pharmaceutically acceptable salt thereof or solvate or solvate of a salt thereof.

There is also provided a method of treating, or reducing the risk of atherosclerosis by changing the composition of the plaques so as to reduce the risk of plaque rupture and atherothrombotic events in a person suffering from or at risk of, said disease or condition, wherein the method comprises administering to the person a therapeutically effective amount of a compound A or a pharmaceutically acceptable salt thereof or solvate or solvate of a salt thereof.

In another aspect the present invention provides a pharmaceutical formulation comprising a therapeutically effective amount of a compound A, or a pharmaceutically acceptable salt thereof or solvate or solvate of a salt thereof, in admixture with a pharmaceutically acceptable adjuvant, diluent or carrier, for use in the treatment or prophylaxis of diseases or conditions in which inhibition of the enzyme MPO is beneficial.

In a further aspect the present invention provides a pharmaceutical formulation comprising a therapeutically effective amount of a compound A, or a pharmaceutically acceptable salt thereof or solvate or solvate of a salt thereof, in admixture with a pharmaceutically acceptable adjuvant, diluent or carrier, for use in the treatment or prophylaxis of neuroinflammatory disorders.

In a further aspect the present invention provides a pharmaceutical formulation comprising a therapeutically effective amount of a compound A, or a pharmaceutically acceptable salt thereof or solvate or solvate of a salt thereof, in admixture with a pharmaceutically acceptable adjuvant, diluent or carrier, for use in the treatment or prophylaxis of multiple sclerosis, cardio- and cerebrovascular atherosclerotic disorders and peripheral artery disease and heart failure and respiratory disorders, such as chronic obstructive pulmonary disease.

In another aspect the present invention provides a pharmaceutical formulation comprising a therapeutically effective amount of a compound A, or a pharmaceutically acceptable salt thereof or solvate or solvate of a salt thereof, in admixture with a pharmaceutically acceptable adjuvant, diluent or carrier, for use in the treatment or prophylaxis of atherosclerosis by preventing and reducing the formation of new atherosclerotic lesions and/or plaques and/or by preventing or slowing progression of existing lesions and plaques.

In another aspect the present invention provides a pharmaceutical formulation comprising a therapeutically effective amount of a compound A, or a pharmaceutically acceptable salt thereof or solvate or solvate of a salt thereof, in admixture with a pharmaceutically acceptable adjuvant, diluent or carrier, for use in the treatment or prophylaxis of atherosclerosis by changing the composition of the plaques so as to reduce the risk of plaque rupture and atherothrombotic events.

The present invention further relates to therapies for the treatment of: Neurodegenerative Disorder(s) including but not limited to Alzheimer's Disease (AD), Dementia, Cognitive Deficit in Schizophrenia (CDS), Mild Cognitive Impairment (MCI), Age-Associated Memory Impairment (AAMI), Age-Related Cognitive Decline (ARCD), Cognitive Impairment No Dementia (CIND), Multiple Sclerosis, Parkinson's Disease (PD), postencephalitic parkinsonism, Huntington's Disease, amyotrophic lateral sclerosis (ALS), motor neuron diseases (MND), Multiple System Atrophy (MSA), Corticobasal Degeneration, Progressive Supranuclear Paresis, Guillain-Barré Syndrome (GBS), and Chronic Inflammatory Demyelinating Polyneuropathy (CIDP). Dementia includes, but is not limited to, Down syndrome, vascular dementia, dementia with Lewy bodies, HIV dementia, Frontotemporal dementia Parkinson's Type (FTDP), Pick's Disease, Niemann-Pick's Disease, traumatic brain injury (TBI), dementia pugilistica, Creutzfeld-Jacob Disease and prion diseases.

The present invention further relates to therapies for the treatment of: Neuroinflammatory Disorder(s) including but not limited to Multiple Sclerosis (MS), Parkinson's disease, Multiple System Atrophy (MSA), Corticobasal Degeneration, Progressive Supranuclear Paresis, Guillain-Barré Syndrome (GBS), chronic inflammatory demyelinating polyneuropathy (CIDP). Multiple sclerosis (MS) includes Relapse Remitting Multiple Sclerosis (RRMS), Secondary Progressive Multiple Sclerosis (SPMS), and Primary Progressive Multiple Sclerosis (PPMS).

The present invention further relates to therapies for the treatment of: Cognitive Disorder(s) including but not limited to

a) Dementia, including but not limited to Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Frontotemporal dementia Parkinson's Type (FTDP), progressive supranuclear palsy (PSP), Pick's Disease, Niemaun-Pick's Disease, corticobasal degeneration, traumatic brain injury (TBI), dementia pugilistica, Creutzfeld-Jacob Disease and prion diseases;

### b) Cognitive Deficit in Schizophrenia (CDS);

### c) Mild Cognitive Impairment (MCI);

### d) Age-Associated Memory Impairment (AAMI);

### e) Age-Related Cognitive Decline (ARCD);

### f) Cognitive Impairment No Dementia (CIND).

The present invention further relates to therapies for the treatment of: Attention-Deficit and Disruptive Behavior Disorder(s) including but not limited to attention deficit disorder (ADD), attention deficit hyperactivity disorder (ADHD) and affective disorders.

The present invention also relates to the treatment of the diseases and conditions below which may be treated with the compounds of the present invention:

respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthiopathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;

The present invention further relates to combination therapies wherein a compound A or a pharmaceutically acceptable salt thereof, or a pharmaceutical composition or formulation comprising a compound A is administered concurrently or sequentially with therapy and/or an agent for the treatment of any one of cardio- and cerebrovascular atherosclerotic disorders and peripheral artery disease.

A compound A or a pharmaceutically acceptable salt thereof may be administered in association with compounds from one or more of the following groups:

1) anti-inflammatory agents, for example
a) NSAIDs (e.g. acetylsalicylic acid, Ibuprofen, naproxen, flurbiprofen, diclofenac, indometacin);
b) leukotriene synthesis inhibitors (5-LO inhibitors e.g. AZD4407, Zileuton, licofelone, CJ13610, CJ13454; FLAP inhibitors e.g. BAY-Y-1015, DG-031, MK591, MK886, A81834; LTA4 hydrolase inhibitors e.g. SC56938, SC57461A);
c) leukotriene receptor antagonists (e.g. CP195543, amelubant, LY293111, accolate, MK571);
   2) anti-hypertensive agents, for example
a) beta-blockers (e.g. metoprolol, atenolol, sotalol);
b) angiotensin converting enzyme inhibitors (e.g. captopril, ramipril, quinapril, enalapril);
c) calcium channel blockers (e.g. verapamil, diltiazem, felodipine, amlodipine);
d) angiotensin II receptor antagonists (e.g. irbesartan, candesartan, telemisartan, losartan);
   3) anti-coagulantia, for example
a) thrombin inhibitors (e.g. ximelagatran), heparines, factor Xa inhibitors;
b) platelet aggregation inhibitors (e.g. clopidrogrel, ticlopidine, prasugrel, AZD 4160);
   4) modulators of lipid metabolism, for example
a) insulin sensitizers such as PPAR agonists (e.g. pioglitazone, rosiglitazone, Galida, muraglitazaar, gefemrozil, fenofibrate);
b) HMG-CoA reductase inhibitors, statins (e.g. simvastatin, pravastatin, atorvastatin, rosuvastatin, fluvastatin);
c) cholesterol absorption inhibitors (e.g. ezetimibe);
d) IBAT inhibitors (e.g. AZD-7806);
e) LXR agonists (e.g. GW-683965A, T-0901317);
f) FXR receptor modulators;
g) phospholipase inhibitors;
   5) anti-anginal agents, for example, nitrates and nitrites;
   6) modulators of oxidative stress, for example, anti-oxidants (e.g. probucol, AGI 1067).

### Methods of Preparation

Another aspect of the present invention provides a process for preparing Compound A or a pharmaceutically acceptable salt thereof.

Throughout the following description of such processes it is understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis” T. W. Green, P. G. M. Wuts, Wiley-Interscience, New York, 1999.

### General Methods

NMR spectra were recorded in DMSO-d₆ as solvent and as internal standard, for ¹H NMR the reference is set to 2.50 ppm and for ¹³C NMR, the reference is set to 39.52 ppm on a Varian Gemini 300 spectrometer operating at 300 MHz for ¹H and 75 MHz for ¹³C, respectively. When solvents are evaporated or reduced in volume, this is performed under reduced pressure. Unless otherwise stated the HPLC analyses were performed on a Gynkotek P580 HPG, gradient pump with a Gynkotek UVD 170S UV-Vis detector. Column: Chromolith Performance RP-18e, 4.6×100 mm, Mobile phase A: Acetonitrile, Mobile phase B: 0.1% TFA (aq), Flow: 3 ml/min, Injection volume: 20 μl, Detection: 220 nm and 254 nm, Gradient: 5% A isocratic for 3 min, 5% A to 100% in 5 minutes. HPLC-purifications were performed on a preparative HPLC, Shimadzu LC-8A, Shimadzu SPD-10A UV-vis.-detector equipped with a Waters X-Terra® prep MS 250 mm×50 mm C18 10 μm. Mobile phase A: Acetonitrile, Mobile phase B: 0.1% TFA (aq), Flow: 100 ml/min, Injection volume: 5 ml, Detection: 254 nm, Gradient: 5% A to 100% in 15 minutes. LC-MS analyses were performed on a Waters 2690 Separations Module with a Waters 2487 Dual X Absorbance Detector and a Waters Micromass ZQ. Column: Chromolith Performance RP-18e, 4.6×100 mm, Mobile phase A: Acetonitrile, Mobile phase B: 0.1% formic acid (aq.), Flow: 2 ml/min, Injection volume: 20 μl, UV-Detection: 254 nm, Gradient: 10% A to 100% in 5 minutes. ZQ with ES−, ES+, MS 97-800, Cone V30 was used.

### WORKING EXAMPLES

### Example 1

### Compound A

### 1-(2-Hydroxyethyl)-2-thioxo-1,2,3,5-tetrahydro-4H-pyrrolo[3,2-d]pyrimidin-4-one

### i) Ethyl 3-{[2-benzyloxy)ethyl]-amino}-1H-pyrrole-2-carboxylate

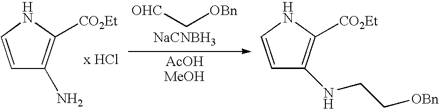

Benzyloxyacetaldehyde (2.84 g, 18.9 mmol) was added dropwise to 3-amino-1H-pyrrole-2-carboxylic acid ethyl ester hydrochloride (3.00 g, 15.7 mmol) (see Furneaux et al, J. Org. Chem. 1999, 64, 8411-8412) and sodium cyanoborhydride (1.00 g, 15.7 mmol) in methanol (60 ml) at room temperature during 5 min. Acetic acid (1.9 g, 31.4 mmol) was added and the reaction mixture was stirred at room temperature for 12 hours. Water (150 ml) was added and the product was extracted with ethylacetate (2×200 ml). The organic phase was evaporated and the remaining yellow oil was used in the next step without further purification. Yield: 4.9 g

¹H NMR (DMSO-d₆) δ 9.85 (1H, s), 7.40-7.20 (6H, m), 6.94 (1H, br s), 6.44 (1H, br s), 4.55 (2H, s), 4.27 (2H, q, J=6.9 Hz), 3.74 (2H, br s), 3.63 (2H, br s) and 1.26 (3H, t, J=6.9 Hz). MS (ESI) m/z 289 (M+1).

### ii) 1-[2-(benzyloxy)ethyl]-2-thioxo-1,2,3,5-tetrahydro-4H-pyrrolo[3,2-d]pyrimidin-4-one

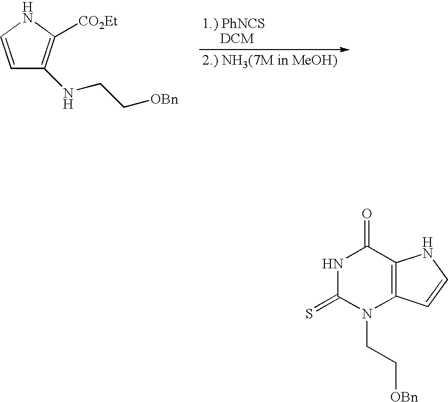

Bensoylisothiocyanate (3.05 g, 18.7 mmol) was added dropwise to ethyl 3-{[2-(benzyloxy)ethyl]amino}-1H-pyrrole-2-carboxylate (4.9 g, 17.0 mmol) in dichloromethane (45 ml) at room temperature for 10 min. The reaction mixture was stirred at room temperature for 30 min. The solvent was evaporated and the remaining yellow oil was dissolved in 7 M NH₃ in methanol (45 ml). The reaction mixture was heated to 80° C. in a closed stainless steel vessel for 2.5 hours. The reaction mixture was cooled to room temperature, ethylacetate (40 mL) was added and the volume was reduced to approximately 25 ml. The precipitate was collected by filtration and the off white crystals were washed with ethylacetate (2×10 ml). Yield: 1.40 g

¹H NMR (DMSO-d₆) δ 12.20 (2H, s), 7.35 (1H, d, J=1.8 Hz), 7.30-7.20 (5H, m), 6.33 (1H, d, J=1.8 Hz), 4.60 (2H, t, J=5.70 Hz), 4.50 (2H, s), 3.83 (3H, t, J=5.70 Hz)

¹³C NMR (DMSO-d₆) δ 172.62, 152.46, 138.19, 137.26, 128.09, 127.63, 127.28, 127.12, 113.61, 97.11, 72.00, 66.14 and 49.55. MS (ESI) m/z 452 (M+1).

### iii) 1-(2-hydroxyethyl)-2-thioxo-1,2,3,5-tetrahydro-4H-pyrrolo[3,2-d]pyrimidin-4-one

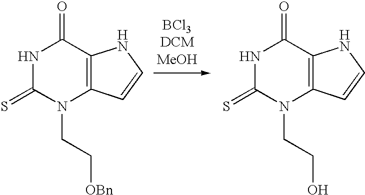

Borontrichloride (1 M in dichloromethane) (13 ml, 13 mmol) was charged in portions to 1-[2-(benzyloxy)ethyl]-2-thioxo-1,2,3,5-tetrahydro-4H-pyrrolo[3,2-d]pyrimidin-4-one (1.33 g, 4.41 mmol) at −78° C. during 5 min. The reaction mixture was stirred at −78° C. for 15 min. Methanol (50 ml) was added, the cooling bath was removed and the solvent was evaporated. The remaining yellow semisolid was purified by preparative HPLC to give 1-(2-hydroxyethyl)-2-thioxo-1,2,3,5-tetrahydro-4H-pyrrolo[3,2-d]pyrimidin-4-one. Yield: 504 mg, 15% over 3 steps.

¹H NMR (DMSO-d₆) δ 12.34 (1H, s), 12.14 (1H, s), 7.34 (1H, t), 6.30 (1H, br s), 4.85 (1H, t, J=5.4 Hz), 4.41 (2H, t, J=6.3 Hz), 3.75 (2H, q, J=6.0 Hz), 1.53-1.43 (2H, m)

¹³C NMR (DMSO-d₆) δ 172.52, 152.52, 137.56, 127.60, 113.62, 97.11, 57.44 25.11.

MS (ESI) m/z 212 (M+1).

### Screens

Methods for the determination of MPO inhibitory activity are disclosed in patent application WO 02/090575. The pharmacological activity of compounds according to the invention was tested in the following screen in which the compound was either tested alone or in the presence of added tyrosine:

Assay buffer: 20 mM sodium/potassium phosphate buffer pH 6.5 containing 10 mM taurine and 100 mM NaCl.

Developing reagent: 2 mM 3,3′,5,5′-tetramethylbenzidine (TMB), 200 μM KI, 200 mM acetate buffer pH 5.4 with 20% DMF.

To 10 μl of diluted compounds in assay buffer, 40 μL of human MPO (final concentration 2.5 nM), with or without 20 μM tyrosine (final concentration, if present, 8 μM), was added and the mixture was incubated for 10 minutes at ambient temperature. Then 50 μl of H₂O₂ (final concentration 100 μM), or assay buffer alone as a control, were added. After incubation for 10 minutes at ambient temperature, the reaction was stopped by adding 10 μl 0.2 mg/ml of catalase (final concentration 18 μg/ml). The reaction mixture was left for an additional 5 minutes before 100 μl of TMB developing reagent was added. The amount of oxidised 3,3′,5,5′-tetramethylbenzidine formed was then measured after about 5 minutes using absorbance spectroscopy at about 650 nM. IC₅₀ values were then determined using standard procedures.

When tested in at least one version of the above screen, the compound of Example 1 gave an IC₅₀ value of less than 5 μM, indicating that it is expected to show useful therapeutic activity.

1. A compound A: 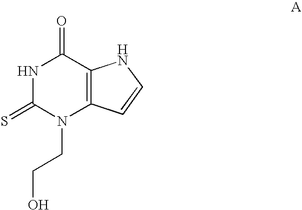
or a pharmaceutically acceptable salt, solvate or solvate of a salt thereof.

2. (canceled)

3. A pharmaceutical composition comprising a compound A, according to claim 1, or a pharmaceutically acceptable salt, solvate or solvate of a salt thereof, optionally in admixture with a pharmaceutically acceptable adjuvant, diluent or carrier.

4. A method of treating, or reducing the risk of, diseases or conditions in which inhibition of the enzyme MPO is beneficial, comprising administering to a person suffering from or at risk of, said disease or condition, a therapeutically effective amount of a compound according to claim 1, or a pharmaceutically acceptable salt, solvate or solvate of a salt thereof.

5. A method of treating, or reducing the risk of inflammatory disorders, comprising administering to a person suffering from or at risk of, said inflammatory disorders, a therapeutically effective amount of a compound according to claim 1, or a pharmaceutically acceptable salt, solvate or solvate of a salt thereof.

6. The method according to claim 5, wherein said inflammatory disorders are neuroinflammatory disorders.

7. The method according to claim 6, wherein said neuroinflammatory disorder is multiple sclerosis.

8. The method according to claim 6, wherein said neuroinflammatory disorder is Parkinson's disease.

9. The method according to claim 5, wherein said inflammatory disorders are selected from cardio- and cerebrovascular atherosclerotic disorders, peripheral artery disease, heart failure, and respiratory disorders.

10. The method according to claim 5, wherein said inflammatory disorder is atherosclerosis.

11. The method according to claim 5, wherein said inflammatory disorder is chronic obstructive pulmonary disease (COPD).

12. The method according to claim 9, wherein said respiratory disorder is selected from bronchitis; infectious bronchitis; eosinophilic bronchitis; emphysema; bronchiectasis; and cystic fibrosis.

13. A method of treating, or reducing the risk of stroke, comprising administering to a person suffering from or at risk of stroke a therapeutically effective amount of compound according to claim 1, or a pharmaceutically acceptable salt, solvate or solvate of a salt thereof.

14. A method of treating, or reducing the risk of, diseases or conditions in which inhibition of the enzyme MPO is beneficial, comprising administering to a person suffering from or at risk of, said disease or condition, a therapeutically effective amount of a pharmaceutical composition according to claim 3.

15-23. (canceled)
